(19) United States
(12) Patent Application Publication
(10) Pub. No.: US 2021/0332386 A1
(43) Pub. Date: Oct. 28, 2021

(54) EXTRACELLULAR VESICLES FOR TARGETED THERAPIES AGAINST MYELOID-DERIVED SUPPRESSOR CELLS

(71) Applicant: Ohio State Innovation Foundation, Columbus, OH (US)

(72) Inventors: Daniel Gallego-Perez, Columbus, OH (US); Silvia Duarte Sanmiguel, Columbus, OH (US); Natalia Higuita-Castro, Columbus, OH (US); William Carson, Hilliard, OH (US)

(21) Appl. No.: 17/284,284

(22) PCT Filed: Oct. 18, 2019
(86) PCT No.: PCT/US2019/057043

§ 371 Date: Apr. 9, 2021

Related U.S. Application Data
(60) Provisional application No. 62/747,982, filed on Oct. 19, 2018.

(51) Int. Cl. C12N 15/88; A61K 9/00; A61K 9/127; C07K 14/705; C12N 15/11; C12N 15/62; A61P 35/00

(52) U.S. Cl. CPC C12N 15/88; A61K 9/0019; A61K 9/1275; A61P 35/00; C12N 15/111; C12N 15/62; C07K 14/70503

(57) ABSTRACT

Disclosed herein are MDSC-targeted extracellular vesicles (EVs) loaded with therapeutic cargo, as well as compositions, systems, and methods for making same. Also disclosed herein is an MDSC-targeting ligand, such as a fusion protein containing an MDSC-targeting moiety. Also disclosed are EVs containing the disclosed fusion protein. In some embodiments, the EV is also loaded with a therapeutic cargo. Also disclosed is an EV-producing cell engineered to produce the disclosed EVs. Also disclosed is a method for making the disclosed EVs that involves culturing the disclosed EV-producing cells under conditions suitable to produce EVs.

### CROSS-REFERENCE TO RELATED APPLICATIONS

This application claims benefit of U.S. Provisional Application No. 62/747,982, filed Oct. 19, 2018, which is hereby incorporated herein by reference in its entirety.

### SEQUENCE LISTING

This application contains a sequence listing filed in electronic form as an ASCII.txt file entitled “321501_2360_Sequence_Listing_ST25” created on Oct. 16, 2019. The content of the sequence listing is incorporated herein in its entirety.

### BACKGROUND

Myeloid-derived suppressor cells (MDSCs) play a fundamental role in a number of physiological and pathological processes, including cancer, wound healing and tissue repair. MDSCs, for example, enable tumor/cancer progression by shielding tumor cells from the host's immune system and/or anti-cancer therapies, and by promoting tumor cell dissemination/spreading. As such, therapeutic approaches need to be developed to selectively target MDSCs and modulate their activity.

### SUMMARY

Disclosed herein is a nanocarrier system to effectively target MDSCs and deliver therapeutic cargo. These nanocarriers are based on designer extracellular vesicles (EVs), which can be autologous (i.e., derived from cells from the same patient) or allogeneic (i.e., derived from cells from a donor) in nature. Cells naturally produce EVs. However to avoid an immune response, cells from the immune system can be used, such as antigen presenting cells (e.g. dendritic cells) or macrophages.

The disclosed ICAM-decorated EVs can be used to target myeloid cells in many different conditions, such as cancer and autoimmune diseases. The decoration cais in some embodiments achieved by transfecting ICAM-expressing vectors into the “donor” cells or tissues. Alternatively, donor cells and tissues can be used that inherently have high levels of ICAM expression.

Designer EVs can therefore be obtained after transfection of cells in vitro, or tissues in vivo, using different transfection techniques (e.g. bulk electroporation, nano-electroporation, tissue nano-transfection, viral transfection, sonoporation, nanoparticles, microparticles, chemical transfection). Loading with therapeutic cargo and/or decoration with MDSC-targeting ligands can be achieved by transfecting the cells with, for example, plasmid DNA encoding ICAM1. ICAM1-based targeting allows for selective EV/cargo delivery to MDSCs within minutes.

Once collected, these EVs can be further modified via electroporation (to add more cargo), biochemical or chemical functionalization to include contrast agents (for diagnostics) or additional targeting or tracing proteins/elements. The EVs could also be further modified with lipid-permeable drugs/chemicals that can enter the EVs via a concentration gradient to further modify the cargo (e.g., to add a pharmacological agent in addition to the genetic cargo).

The therapeutic cargo could be varied depending on whether myelosuppresor activity is to be enhanced or tamed, depending on the condition that is being treated. For example, loading ICAM1-decorated EVs with miR146a, for example, could be used to counter MDSC activity within the tumor niche. In addition to nucleic acid-based therapeutic cargo, ICAM1-decorated EVs can be loaded with membrane-permeable pharmacological compounds (e.g., Ibrutinib), which can diffuse into the EVs via a concentration gradient. In some embodiments, the cargo is an imaging agent, such as a contrast agent, for diagnostic imaging.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of production of ICAM1-decorated EVs to target MDSCs. EVs are made by delivering plasmids encoding for ICAM1 and therapeutic cargo (for nucleic acid-based therapeutics) into autologous or allogeneic cells (in vitro or in vivo). The cell machinery process these plasmids to then enable the production of designer EVs decorated with ICAM1 and loaded with the nucleic acid of interest.

FIG. 2 is a schematic representation of surface-decorated EVs loaded with pharmacotherapeutic cargo for example, Ibrutinib, a membrane-permeable pharmacological compound that inhibits brutontyrosine kinase (BTK).

FIG. 3 illustrates ICAM1-decorated EVs can be used to target MDSCs and Tumor associated macrophages (TAMs) within the tumor microenvironment to counter their immunosuppressive activity and facilitate the treatment of the tumor.

FIG. 4A shows ICAM1-decorated EVs were preferentially internalized by MDSCs or macrophages (e.g., TAMs) and not cancer cells (A549) after 15 min of incubation (EVs were labeled with a green fluorescent dye). FIG. 4B shows loading of miR-146a in decorated EVs. Scr-CT are control (CT) EVs made by transfecting cells with a scrambled plasmid.

FIG. 5 is a bar graph showing EV-based treatment hinders tumor growth.

FIGS. 6A and 6B are bar graphs showing EV-based treatment impacts the immune cell make-up of the tumor (FIG. 6A) and reduces MDSCs (FIG. 6B).

FIG. 7 illustrates an experiment to validate engineered EVs

FIGS. 8A and 8B show the levels of miR146a found in the loaded EVs increased ˜400-fold (FIG. 8A) and the levels of GLUT-1 increased ˜3000-fold compared to control EVs (FIG. 8B). FIG. 8C is a western blot showing that the EVs were decorated with ICAM-1.

FIG. 9A shows ICAM-decorated EVs target MDSCs. We co-cultured MDCSs and cancer cells and we treated them with EEVs during 72 h. FIG. 9B shows that MDSCs switched to a proinflammatory phenotype.

FIG. 10 shows engineered EVs reduce tumor progression in a murine model of breast cancer (PyMT).

FIGS. 11A and 11B show engineered EVs display immunomodulatory activity. Tumors injected with engineered EVs had less monocytic MDSCs compared to baseline (FIG. 11) but no change in macrophages (FIG. 11B).

FIGS. 12A and 12B show engineered EVs have increased T cell infiltration.

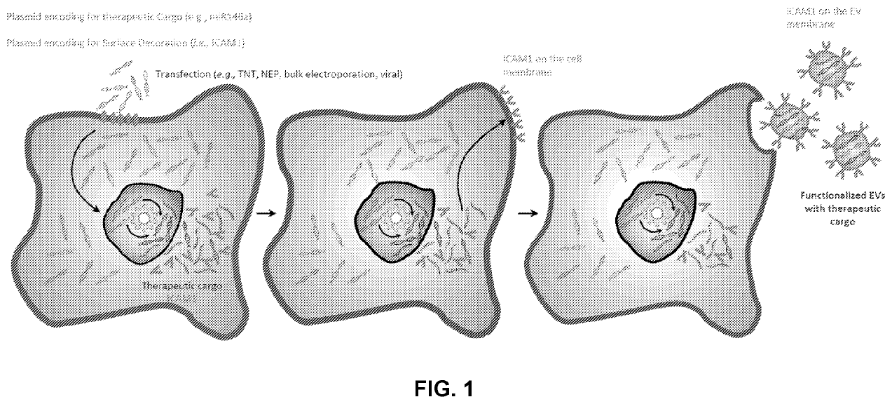

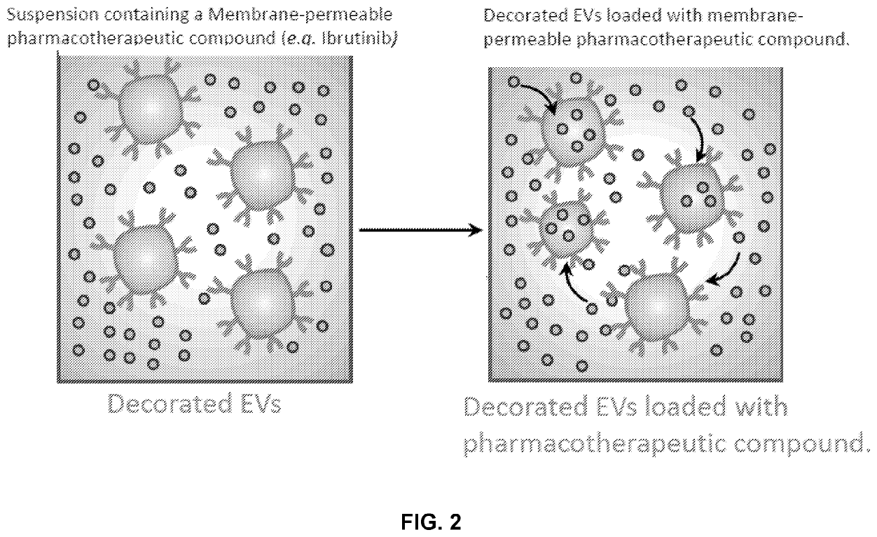

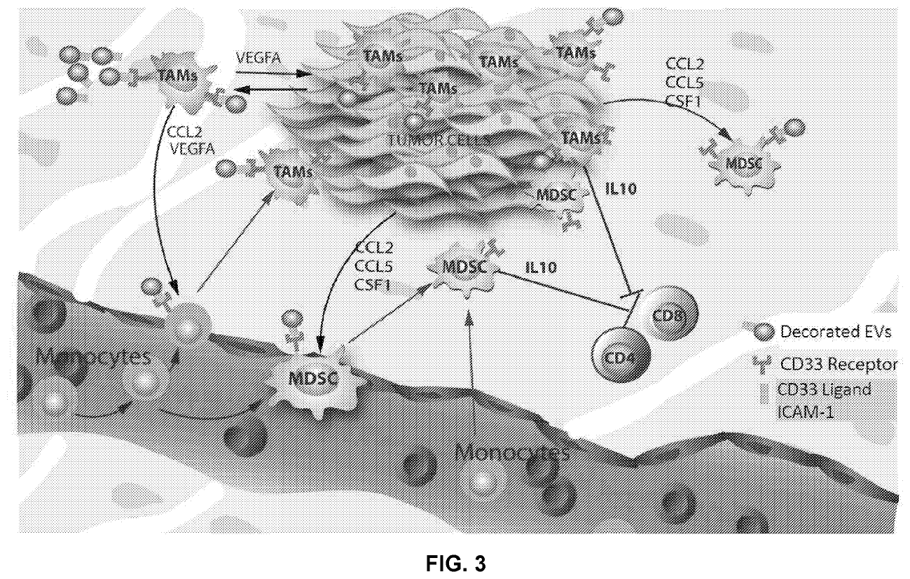

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Disclosed are MDSC-targeted extracellular vesicles (EVs) loaded with therapeutic cargo, as well as compositions, systems, and methods for making same. Also disclosed herein is an MDSC-targeting ligand, such as a fusion protein containing an MDSC-targeting moiety. Also disclosed are EVs containing the disclosed fusion protein. In some embodiments, the EV is also loaded with a therapeutic cargo. Also disclosed is an EV-producing cell engineered to produce the disclosed EVs. Also disclosed is a method for making the disclosed EVs that involves culturing the disclosed EV-producing cells under conditions suitable to produce EVs. The method can further involve purifying EVs from the cell.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Extracellular Vehicles (EVs)

The disclosed EVs can in some embodiments be any vesicle that can be secreted by a cell. Cells secrete extracellular vesicles (EVs) with a broad range of diameters and functions, including apoptotic bodies (1-5 μm), microvesicles (100-1000 nm in size), and vesicles of endosomal origin, known as exosomes (50-150 nm).

The disclosed extracellular vesicles may be prepared by methods known in the art. For example, the disclosed extracellular vesicles may be prepared by expressing in a eukaryotic cell an mRNA that encodes the cell-targeting ligand. In some embodiments, the cell also expresses an mRNA that encodes a therapeutic cargo. The mRNA for the cell-targeting ligand and the therapeutic cargo may be expressed from vectors that are transfected into suitable production cells for producing the disclosed EVs. The mRNA for the cell-targeting ligand and the therapeutic cargo may be expressed from the same vector (e.g., where the vector expresses the mRNA for the cell-targeting ligand and the therapeutic cargo from separate promoters), or the mRNA for the cell-targeting ligand and the therapeutic cargo may be expressed from separate vectors. The vector or vectors for expressing the mRNA for the cell-targeting ligand and the therapeutic cargo may be packaged in a kit designed for preparing the disclosed extracellular vesicles.

Also disclosed is a composition comprising an EV containing the disclosed targeting ligands. In some embodiments, the EV is loaded with a disclosed therapeutic cargos. Also disclosed is an EV-producing cell engineered to secrete the disclosed EVs.

EVs, such as exosomes, are produced by many different types of cells including immune cells such as B lymphocytes, T lymphocytes, dendritic cells (DCs) and most cells. EVs are also produced, for example, by glioma cells, platelets, reticulocytes, neurons, intestinal epithelial cells and tumor cells. EVs for use in the disclosed compositions and methods can be derived from any suitable cell, including the cells identified above. Non-limiting examples of suitable EV producing cells for mass production include dendritic cells (e.g., immature dendritic cell), Human Embryonic Kidney 293 (HEK) cells, 293T cells, Chinese hamster ovary (CHO) cells, and human ESC-derived mesenchymal stem cells. EVs can also be obtained from autologous patient-derived, heterologous haplotype-matched or heterologous stem cells so to reduce or avoid the generation of an immune response in a patient to whom the exosomes are delivered. Any EV-producing cell can be used for this purpose.

Also disclosed is a method for making the disclosed EVs loaded with a therapeutic cargo that involves culturing the disclosed EV-producing cell engineered to secrete the disclosed EVs. The method can further involves purifying EVs from the cells.

EVs produced from cells can be collected from the culture medium by any suitable method. Typically a preparation of EVs can be prepared from cell culture or tissue supernatant by centrifugation, filtration or combinations of these methods. For example, EVs can be prepared by differential centrifugation, that is low speed (<20000 g) centrifugation to pellet larger particles followed by high speed (>100000 g) centrifugation to pellet EVs, size filtration with appropriate filters, gradient ultracentrifugation (for example, with sucrose gradient) or a combination of these methods.

### MDSC-Targeting Ligands

The disclosed EVs can be targeted to MDSCs by expressing on the surface of the EVs a targeting moiety which binds to a cell surface moiety expressed on the surface of the MDSCs. Examples of suitable targeting moieties are short peptides, scFv and complete proteins, so long as the targeting moiety can be expressed on the surface of the exosome. Peptide targeting moieties may typically be less than 100 amino acids in length, for example less than 50 amino acids in length, less than 30 amino acids in length, to a minimum length of 10, 5 or 3 amino acids.

For example, in some embodiments, the cell targeting ligand is ICAM1. ICAM1-based targeting allows for selective EV/cargo delivery to MDSCs within minutes. In some embodiments, the targeting ligand is ICAM-1 and has the amino acid sequence: MASTRAKPTLPLLLALVTVVIPGPGDAQVSIHPREAFLPQGGSVQVNCSSSCKEDLSLGLET QWLKDELESGPNWKLFELSEIGEDSSPLCFENCGTVQSSASATITVYSFPESVELRPLPAW QQVGKDLTLRCHVDGGAPRTQLSAVLLRGEEILSRQPVGGHPKDPKEITFTVLASRGDHGA NFSCRTELDLRPQGLALFSNVSEARSLRTFDLPATIPKLDTPDLLEVGTQQKLFCSLEGLFP ASEARIYLELGGQMPTQESTNSSDSVSATALVEVTEEFDRTLPLRCVLELADQILETQRTLT VYNFSAPVLTLSQLEVSEGSQVTVKCEAHSGSKVVLLSGVEPRPPTPQVQFTLNASSEDHK RSFFCSAALEVAGKFLFKNQTLELHVLYGPRLDETDCLGNWTWQEGSQQTLKCQAWGNP SPKMTCRRKADGALLPIGVVKSVKQEMNGTYVCHAFSSHGNVTRNVYLTVLYHSQNNWTll ILVPVLLVIVGLVMAASYVYNRQRKIRIYKLQKAQEEAIKLKGQAPPP (SEQ ID NO:1), or a variant and/or fragment thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO:1 that can target a molecule on MDSCs. For example, the targeting ligand can be a fragment and/or variant of SEQ ID NO:1 capable of binding the amino acid sequence LYQAKRFKV (SEQ ID NO:2), which can in some embodiments define an ICAM-1-binding site.

In some embodiments, the targeting ligand is a fragment of ICAM-1 comprising at least 100, 110, 120, 130, 140, 141, 142, 143, 144, 145, 156, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 420, 430, 440, 441, 442, 443, 444, 445, 456, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, or 537 contiguous amino acids of SEQ ID NO:1 or a variant thereof.

The binding sites in ICAM-1 are described, for example, in Diamond M S, et al. Cell 1991 65:961-971, and Hermand P, et al. J Biol Chem 2000 275(34):26002-26010, which are incorporated by reference in their entireties for the teaching of these binding domains.

In some embodiments, the cell targeting ligand can be expressed on the surface of the EV by expressing it as a fusion protein with an exosomal or lysosomal transmembrane protein.

### Therapeutic Cargo

The disclosed extracellular vesicles further may be loaded with a therapeutic agent, where the extracellular vesicles deliver the agent to a target cell. Suitable therapeutic agents include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA (also referred to herein as a “cargo RNA”).

For example, in some embodiments the fusion protein containing the cell-targeting motif also includes an RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. As such, the fusion protein may function as both of a “cell-targeting protein” and a “packaging protein.” In some embodiments, the packaging protein may be referred to as extracellular vesicle-loading protein or “EV-loading protein.”

In some embodiments, the cargo RNA is an miRNA, shRNA, mRNA, ncRNA, sgRNA or any combination thereof. For example, in some embodiments, the anti-inflammatory agent is micro-RNA 146a. Other miRNAs have been reported to regulate the expression of key molecules responsible for M1-favoring glycolytic metabolism (e.g., mRr9, miR127 and miR155).

The cargo RNA of the disclosed extracellular vesicles may be of any suitable length. For example, in some embodiments the cargo RNA may have a nucleotide length of at least about 10 nt, 20 nt, 30 nt, 40 nt, 50 nt, 100 nt, 200 nt, 500 nt, 1000 nt, 2000 nt, 5000 nt, or longer. In other embodiments, the cargo RNA may have a nucleotide length of no more than about 5000 nt, 2000 nt, 1000 nt, 500 nt, 200 nt, 100 nt, 50 nt, 40 nt, 30 nt, 20 nt, or 10 nt. In even further embodiments, the cargo RNA may have a nucleotide length within a range of these contemplated nucleotide lengths, for example, a nucleotide length between a range of about 10 nt-5000 nt, or other ranges. The cargo RNA of the disclosed extracellular vesicles may be relatively long, for example, where the cargo RNA comprises an mRNA or another relatively long RNA.

In some embodiments, the therapeutic cargo is a membrane-permeable pharmacological compound that is loaded into the EV after it is secreted by the cell. In some embodiments, the cargo is an anti-cancer agent that can cause apoptosis or pyroptosis of a targeted tumor cell. In some embodiments, the anti-cancer agent is a small molecule drug. For example, in some embodiments, the cargo is Ibrutinib. Additional examples of anti-cancer drugs or antineoplastics to be attached to the tumor targeting peptides described herein include, but are not limited to, aclarubicin, altretamine, aminopterin, amrubicin, azacitidine, azathioprine, belotecan, busulfan, camptothecin, capecitabine, carboplatin, carmofur, carmustine, chlorambucil, cisplatin, cladribine, clofarabine, cyclophosphamide, cytarabine, daunorubicin, decitabine, doxorubicin, epirubicin, etoposide, floxuridine, fludarabine, 5-fluorouracil, fluorouracil, gemcitabine, idarubicin, ifosfamide, irinotecan, mechlorethamine, melphalan, mercaptopurine, methotrexate, mitoxantrone, nedaplatin, oxaliplatin, paclitaxel, pemetrexed, pentostatin, pirarubicin, pixantrone, procarbazine, pyrimethamine raltitrexed, rubitecan, satraplatin, streptozocin, thioguanine, triplatin tetranitrate, teniposide, topotecan, tegafur, trimethoprim, uramustine, valrubicin, vinblastine, vincristine, vindesine, vinflunine, vinorelbine, and zorubicin.

To achieve loading of small RNAs into EVs, transfection-based approaches have been proposed. Other reports have shown that using vector-induced expression of small RNAs in cells, small RNA loading into EVs can be achieved. Alternatively, EV donor cells may be transfected with small RNAs directly. Incubation of tumor cells with chemotherapeutic drugs is also another method to package drugs into EVs. To stimulate formation of drug-loaded EVs, cells are irradiated with ultraviolet light to induce apoptosis. Alternative approaches such as fusogenic liposomes also leads loading drugs into EVs.

In some embodiments, the therapeutic cargo is loaded into the EVs by diffusion via a concentration gradient.

### Methods

Also contemplated herein are methods for using the disclosed EVs. For example, the disclosed extracellular vesicles may be used for delivering the disclosed therapeutic cargo to myeloid-derived suppressor cells (MDSCs), where the methods include contacting the target cell with the disclosed EVs.

MDSCs play a fundamental role in a number of physiological and pathological processes, including cancer, wound healing and tissue repair. The disclosed EVs may be formulated as part of a pharmaceutical composition for treating a disease or disorder involving MDSCs and the pharmaceutical composition may be administered to a patient in need thereof to deliver the cargo to target MDSCs in order to treat the disease or disorder. The fore, also disclosed herein is a method of treating a disease involving MDSCs in a subject, that involves administering to the subject a therapeutically effective amount of a composition containing cargo-loaded MDSC-targeted EVs disclosed herein. In some embodiments, the subject has cancer. In some embodiments, the subject as detectable circulating MDSCs. Recent studies have demonstrated that MDSCs can be involved in many pathological conditions such as bacterial, viral and parasitic infections, traumatic stress, sepsis, acute inflammation, graft versus host disease and different autoimmune diseases like diabetes, encephalomyelitis and colitis.

The disclosed EVs may be administered to a subject by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

Parenteral administration is generally characterized by injection, such as subcutaneously, intramuscularly, or intravenously. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone.

Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration should be sterile, as is known and practiced in the art.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection.

Preferably, the dose of a single intramuscular injection is in the range of about 5 to 20 μg. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

A number of embodiments of the invention have been described. Nevertheless, it will be understood that various modifications may be made without departing from the spirit and scope of the invention. Accordingly, other embodiments are within the scope of the following claims.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

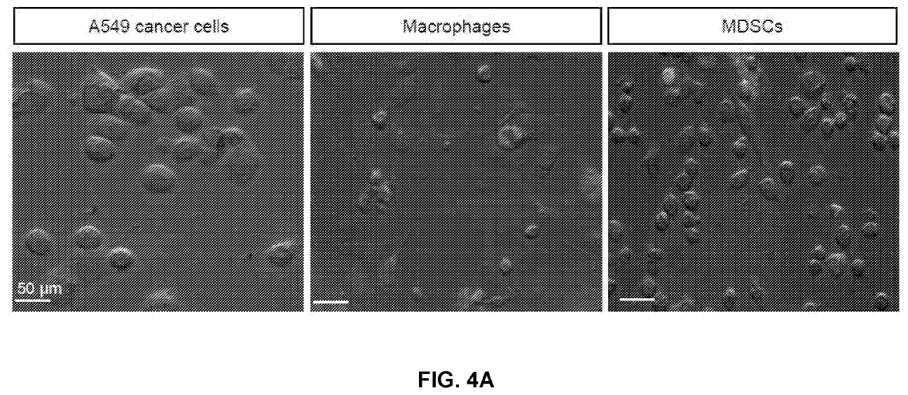

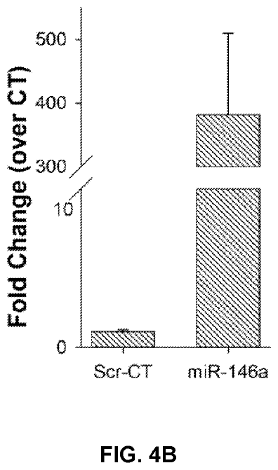

DCs were nanotransfected with plasmids for miR146a. EVs were isolated from the culture media using an ExoQuick. To evaluate the in vitro efficacy, in vitro monoculture was used with MDSCs or macrophages and not cancer cells (A549). Cells were treated with cargo EVs. FIG. 4A shows ICAM1-decorated EVs were preferentially internalized by MDSCs or macrophages (e.g., TAMs) and not cancer cells (A549) after 15 min of incubation (EVs were labeled with a green fluorescent dye). rq-PCR for miR146a was performed. FIG. 4B shows loading of miR-146a in decorated EVs. Scr-CT are control (CT) EVs made by transfecting cells with a scrambled plasmid. +

### Example 2

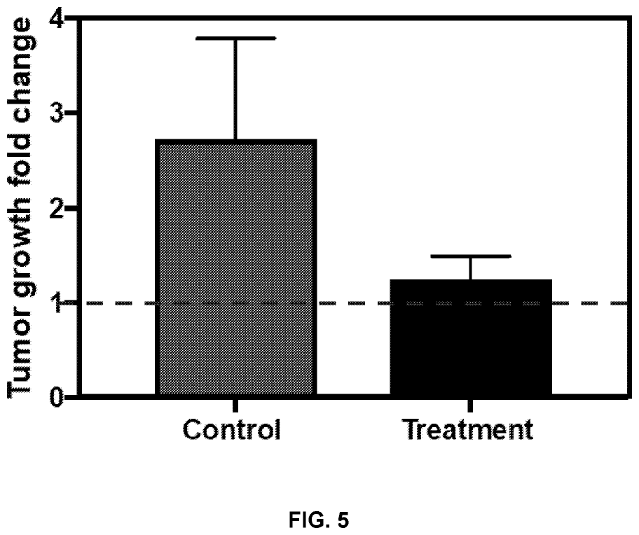

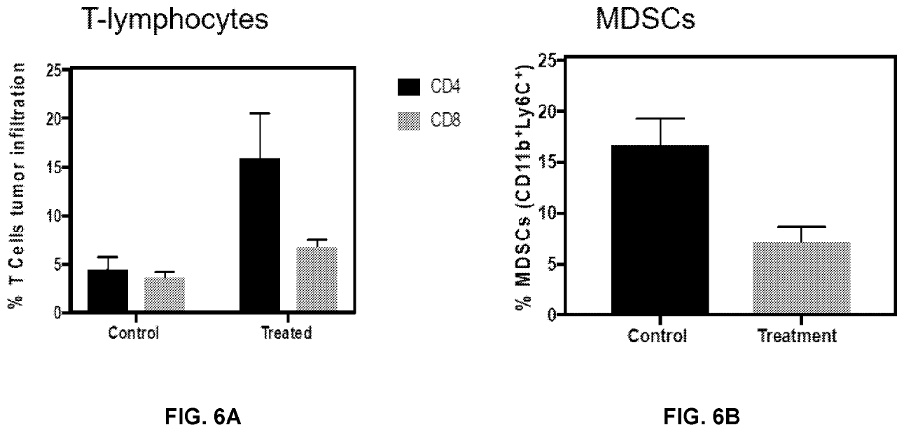

Designer EVs for targeted therapies against myeloid-derived suppressor cells hinder tumor growth (FIG. 5). As shown in FIGS. 6A and 6B, EV-based treatment impacts the immune cell make-up of the tumor by increasing CD4 and CD8 cells and reduces MDSCs (FIG. 6B).

### Example 3

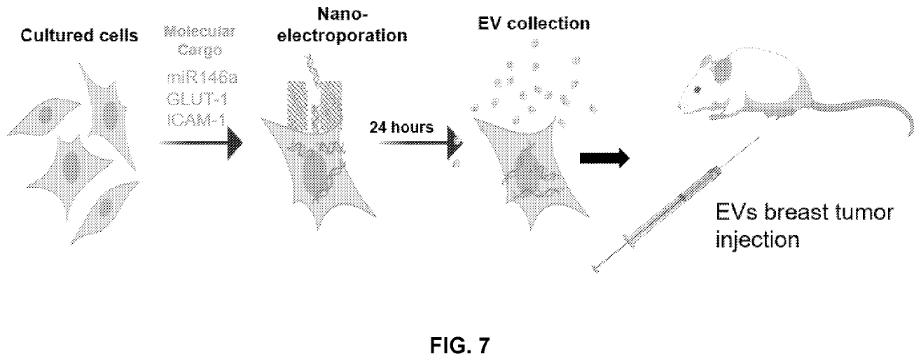

Direct injection of EVs generated by TNT to promote immunomodulation of the tumor environment. In order to show that the EVs were indeed responsible of the decrease of the tumor progression, EVs were fabricated in vitro using nano-electrophoration, which were then loaded with miR146a, GLUT-1, and ICAM-1, referred to as “engineered EVs” (FIG. 7) Engineered EVs were produced from embryonic fibroblast as a skin model.

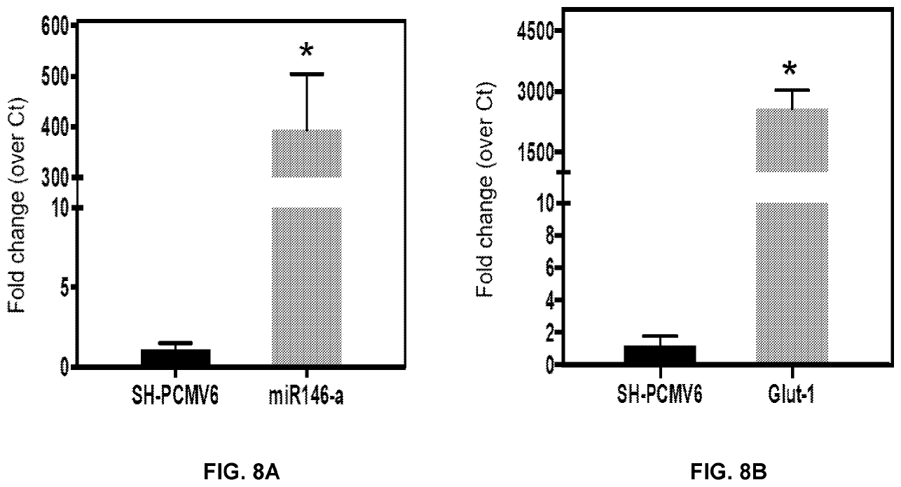

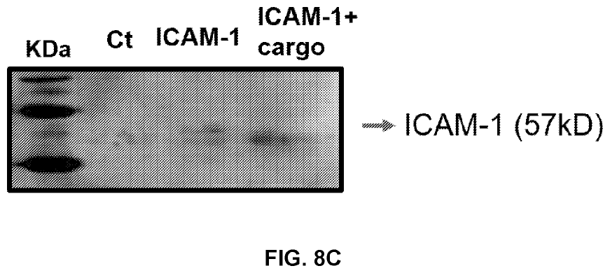

When engineered EVs were loaded with the cargo of interest, the levels of miR146a found in the loaded EVs increased approximately 400-fold and the levels of GLUT-1 increased approximately 3000-fold compared to control EVs (FIGS. 8A and 8B). FIG. 8C is a western blot showing that the EVs were decorated with ICAM-1.

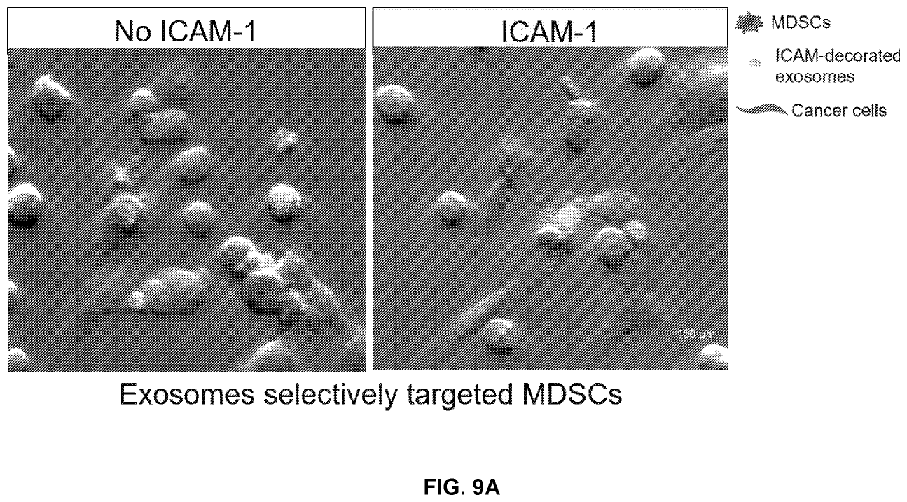

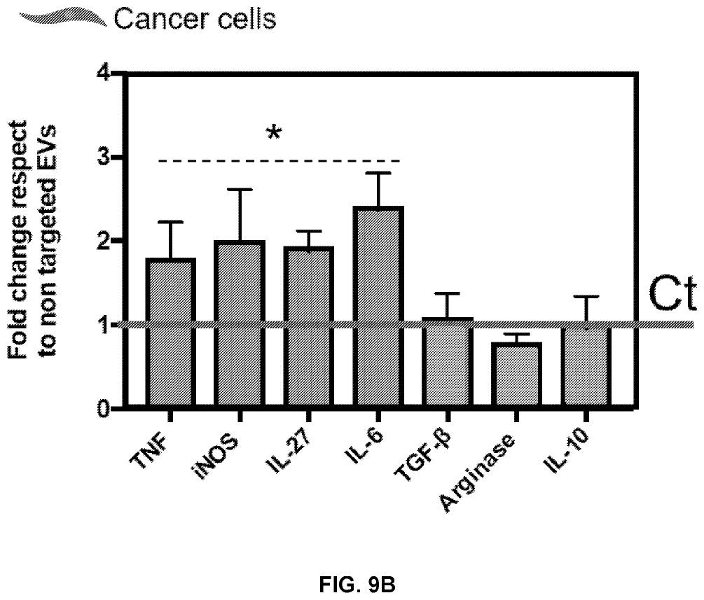

In order to test whether the EEVs targeted MDSCs, MDCSs and cancer cells were co-cultured and treated with engineered EVs during 72 h. When the EVs were not decorated, they were taken by both MDSCs and tumor cells. However, decorated engineered EVs selectively targeted the MDSCs and not the tumor cell (FIG. 9A). In addition, MDSCs switched to a proinflammatory phenotype: there was an increase in the levels of proinflammatory markers, and a decrease in the levels of the anti-inflammatory or tumor protecting markers (FIG. 9B).

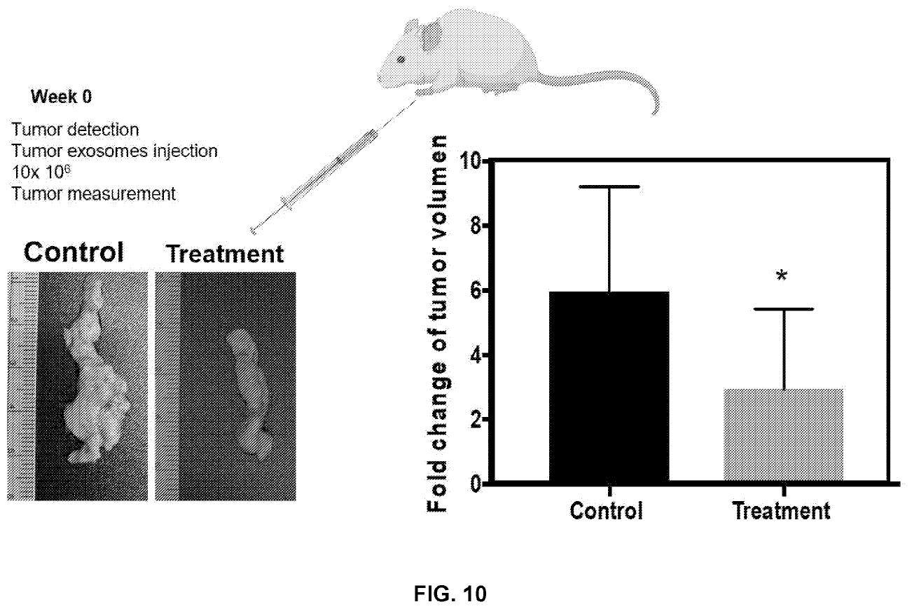

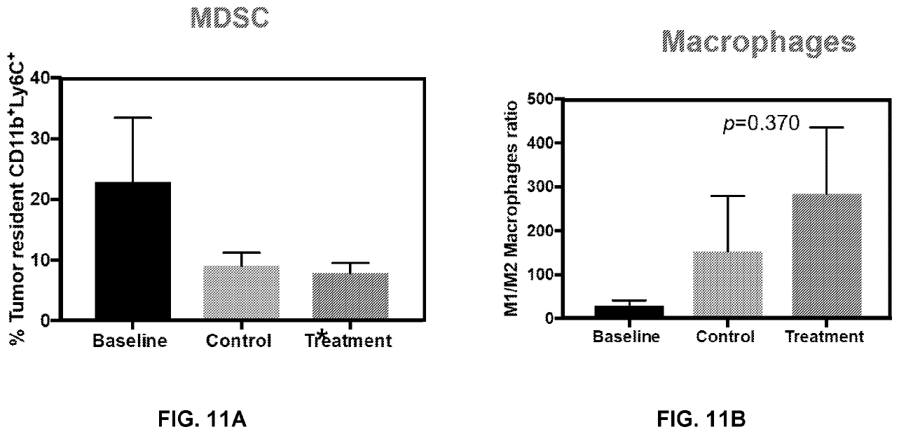

The functional engineered EVs were injected directly into the tumor of a murine model of breast cancer (PyMT), causing tumor progression to be reduced in treated animals (FIG. 10). As shown in FIGS. 11A and 11B, tumors injected with engineered EVs had less monocytic MDSCs compared to baseline (FIG. 11A). These results suggest that engineered EVs can be used to increase the proportion of pro-inflammatory myeloid cells in the tumor.

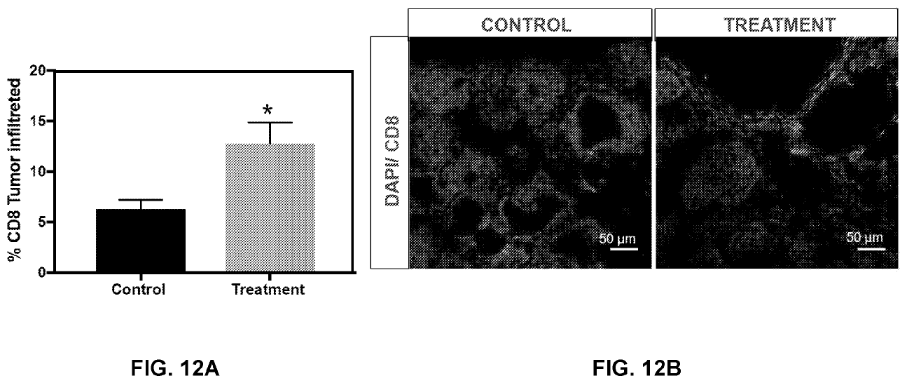

Finally, the injection of engineered EVs clearly promoted increased infiltration by cytotoxic T cells compared to controls (FIGS. 12A and 12B). So all together, these results confirm that when EVs are injected, it makes the tumor “hotter” or more proinflammatory

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A fusion protein comprising ICAM1 and an exosomal or lysosomal transmembrane protein.

2. The extracellular vesicle comprising the fusion protein of claim 1.

3. The extracellular vesicle of claim 2, further comprising a therapeutic cargo.

4. The extracellular vesicle of claim 3, wherein the therapeutic cargo comprises miR146a.

5. The extracellular vesicle of claim 3, wherein the therapeutic cargo comprises Ibrutinib.

6. A cell comprising a nucleic acid encoding the fusion protein of claim 1.

7. The cell of claim 6, further comprising a nucleic acid encoding a therapeutic RNA.

8. A method of producing an extracellular vesicle, comprising culturing the cell of claim 6 under conditions suitable for vesicle secretion, and isolating extracellular vesicles secreted by the cell.

9. The method of claim 8, further comprising loading the extracellular vesicle with a therapeutic drug.

10. A method of treating cancer in a subject, comprising administering to the subject a therapeutically effective amount of the extracellular vesicle of claim 3.

11. The method of claim 10, wherein the subject has circulating myeloid-derived suppressor cells (MDSCs).


SEQUENCE LISTING (2 sequences)

SEQ ID NO: 1 - protein, 537 aa, Homo sapiens
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 2 - protein, 9 aa, Homo sapiens
LYQAKRFKV